(19) United States
(12) Patent Application Publication
(10) Pub. No.: US 2010/0209488 A1
(43) Pub. Date: Aug. 19, 2010

(54) PROTEIN KINASE MODULATING COMPOUNDS AND METHODS FOR MAKING AND USING THEM

(75) Inventors: Wolfgang Wrasidlo, La Jolla, CA (US); Eric Murphy, San Marcos, CA (US); David Cheresh, Encinitas, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, Oakland, CA (US)

(21) Appl. No.: 12/602,759

(22) PCT Filed: Jul. 15, 2008
(86) PCT No.: PCT/US08/70079

§ 371 Date: May 3, 2010

Related U.S. Application Data
(60) Provisional application No. 60/950,104, filed on Jul. 16, 2007.

(51) Int. Cl. A61K 31/5377; C07D 413/14; A61K 9/127; A61K 9/14; C12N 9/99; A61P 35/00; A61P 9/00; A61P 25/00; A61P 3/10; A61P 29/00; A61P 9/12; A61P 35/02; A61P 35/04

(52) U.S. Cl. 424/450; 544/139; 514/235.8; 544/123; 424/489; 435/184

(57) ABSTRACT

The invention provides compositions, e.g., small molecules, that disrupt the activity of protein kinases, including constitutively activated protein kinases, such as kinases constitutively activated through mutations, and kinases in their native inactivated state, and methods for making and using them. In one aspect, compositions of the invention bind a protein kinase in its inactive conformation involving the conserved asparagine-phenylalanine-glycine residue motif, or “Asp-Phe-Gly” or “DFG” motif, of the activation loop in the allosteric binding site. The small molecule protein kinase inhibitors of the invention comprise or are derivatives or analogs of oxadiazoles, thiadiazoles, oxazoles, thiazoles, arylamides, quinolones, pyrazoles, pyrazolones, imides, pyrolles, imidazoles and/or triazoles.

### TECHNICAL FIELD

This invention generally relates to small molecule chemistry, biochemistry, cell and tumor biology and medicine. The invention provides compositions, e.g., small molecules, that disrupt (e.g., inhibit) the activity of protein kinases, including constitutively activated protein kinases, such as kinases constitutively activated through mutations, and kinases in their native inactivated state, and methods for making and using them. In one aspect, compositions of the invention bind a protein kinase in its inactive conformation involving the conserved asparagine-phenylalanine-glycine residue motif, or “Asp-Phe-Gly” or “DFG” motif, of the activation loop in the allosteric binding site. The small molecule protein kinase inhibitors of the invention comprise or are derivatives or analogs of oxadiazoles, thiadiazoles, oxazoles, thiazoles, arylamides, quinolones, pyrazoles, pyrazolones, imides, pyrolles, imidazoles and/or triazoles, as described herein.

### BACKGROUND

Abnormal signaling of protein kinases, either through mutations, phosphorylation, or protein-protein interactions in cells, can lead to cellular aberrations associated with a variety of disease states, e.g., cancer or diseases of the vasculature or neurological disorders. Constitutively activated protein kinases can lead to uncontrolled cell proliferation, such as in cancer.

Protein kinase inhibitors that function by blocking ATP binding sites or inhibitors which block the allosteric binding site of kinases relatively nonspecifically have been described. The existing art takes advantage of inhibiting the ATP binding site of kinases (ATP mimetics) to block kinase activity in active protein kinases. Recently some inhibitors (e.g., Bay-43-9006, or SORAFENIB™, Bayer Pharmaceuticals, and imatinib mesylate, or GLEEVEC™, Novartis) target the allosteric binding site of these protein kinases. Although useful compounds can be found using either approach, most compounds lack a high degree of specificity.

### SUMMARY

The invention provides compositions, e.g., small molecules, that disrupt (e.g., inhibit, diminish) the activity of protein kinases, including constitutively activated protein kinases, and methods for making and using them. In one aspect, compositions of the invention bind a protein kinase in an inactive conformation, thus inhibiting its activity. In one aspect, compositions of the invention bind a protein kinase in its asparagine-phenylalanine-glycine residue motif, or “Asp-Phe-Gly” or “DFG” motif of the activation loop in the allosteric binding site. The small molecule protein kinase inhibitors of the invention comprise (or consist of) oxadiazoles, thiodiazoles, oxazoles, thiazoles, arylamides, quinolones, pyrazoles, pyrazolones, imides, pyrolles, imidazoles and/or triazoles, or comprise (or consist of) derivatives or analogs of oxadiazoles, thiodiazoles, oxazoles, thiazoles, arylamides, quinolones, pyrazoles, pyrazolones, imides, pyrolles, imidazoles and/or triazoles, as described herein.

The invention provides compounds having (comprising)

(a) formula (I)

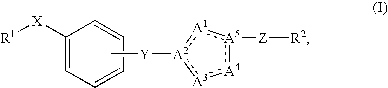

wherein R¹ is an optionally substituted 5-6 membered heterocyclic ring;

R² is a 3-7 membered alicyclic ring or a 5-6 membered aromatic ring, each of which may be optionally substituted;

X is CH₂, O, S, SO, SO₂, NH or C═O;

Y is a bond or NH, O or S;

Z is a bond, NH, O, S, NH(C═O), or NHSO₂;

A¹ is CH, NH, CH₂, C═O, O or S; and

A², A³, A⁴, and A⁵ are independently C, CH, NH or N; or

(b) a pharmaceutically acceptable salt of (a).

In alternative embodiments of these compounds of the invention, R¹ is selected from the group consisting of
pyridinyl, pyrazinyl, pyrimidinyl, pyridazinyl, triazinyl,
pyrrolyl, pyrazolyl, imidazolyl, triazolyl, pyrazolinyl,
piperidinyl, piperazinyl, pyrrolidinyl,
furanyl, pyranyl, tetrahydrofuranyl, dioxanyl, thiophenyl,

oxazolyl, isoxazolyl, oxadiazolyl, thiazolyl, isothiazolyl, thiadiazolyl, morpholinyl, and

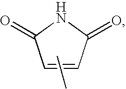

each of which is optionally substituted with one or more substituents. In alternative embodiments of these compounds of the invention, the substituents can be (can comprise) C1-C6 alkyl, C2-C6 alkenyl, C2-C6 alkynyl, C1-C6 heteroalkyl, C2-C6 heteroalkenyl, C2-C6 heteroalkynyl, C1-C6 acyl, C1-C6 heteroacyl, halo, CN, NO₂, C═O, NH₂, and SO₂.

In alternative embodiments, substituents also can be (can comprise) C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or it can be halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂, wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl, and wherein two R can be linked to form a 3-8 membered ring, optionally containing one or more N, O or S; and wherein each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O; and wherein two R′ can be linked to form a 3-7 membered ring optionally containing up to three heteroatoms selected from N, O and S;

In alternative embodiments of these compounds of the invention, R² is a 5 to 6 membered aromatic ring, or a 4, 5, 6, or 7 membered ring, wherein the aromatic ring is phenyl, pyrrolyl, thiophenyl, furanyl, pyridinyl or pyranyl.

In alternative embodiments of these compounds of the invention, A², A³ and A⁴ are N. In alternative embodiments of these compounds of the invention, A¹ is CH and A⁵ is C.

In alternative embodiments of these compounds of the invention, the compound is comprised of (comprises) at least one, two, three, four, five or six rings.

In alternative embodiments of these compounds of the invention, R² is selected from the group consisting of phenyl, cyclopentadiene, and C3-C6 cycloalkyl, each of which is optionally substituted.

In alternative embodiments of these compounds of the invention, R² is phenyl, wherein phenyl is substituted with:

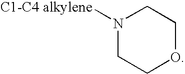

In alternative embodiments of these compounds of the invention, the compound comprises the formula (depicted as)

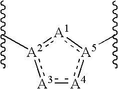

is selected from the group consisting of

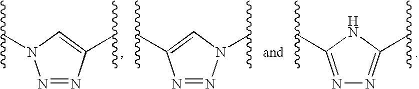

In alternative embodiments of these compounds of the invention, X is O; or, R¹ is imidazolyl or pyrimidinyl.

The invention provides compounds selected from the group consisting of

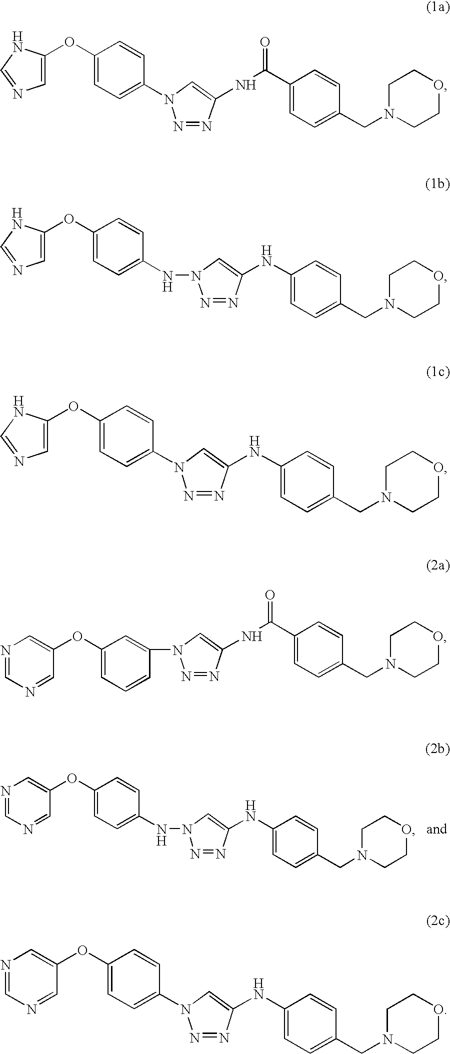

The invention provides pharmaceutical compositions comprising one or more compounds of the invention.

The invention provides pharmaceutical formulations and pharmaceutical compositions comprising:

(a) one or more compounds of the invention;

(b) the pharmaceutical formulation or pharmaceutical composition of (a), comprising (formulated with) a cyclodextrin or a cycloamylose;

(c) the pharmaceutical formulation or pharmaceutical composition of (a) or (b), formulated as a sterile injectable aqueous solution, or an oleaginous suspension;

(d) the pharmaceutical formulation or pharmaceutical composition of any of (a) to (d), formulated with a (comprising a) diluent, an emulsifier, a preservative, a buffer, a pharmaceutically acceptable excipient, or a combination thereof;

(e) the pharmaceutical formulation or pharmaceutical composition of any of (a) to (d), formulated as a liquid, an emulsion, a lyophilized powder, a spray, a cream, a lotion, a controlled release formulation, a tablet, a pill, a gel, a patch, in an implant or in a spray.

The invention provides liposomes, a nanoparticles and/or microspheres comprising one or more compounds of the invention, or one or more pharmaceutical compositions of the invention, or one or more pharmaceutical formulations of the invention.

The invention provides uses of any (one or more) compound of the invention, or any (one or more) pharmaceutical composition of the invention, or any (one or more) pharmaceutical formulation of the invention, for the manufacture of a medicament.

The invention provides uses of any (one or more) compound of the invention, or any (one or more) pharmaceutical composition of the invention, or any (one or more) pharmaceutical formulation of the invention, for the manufacture of a medicament for the treatment, prevent or amelioration of diseases or conditions associated with dysfunctional cells, cancer stem cells or cancer cells activated by a DFG motif-comprising protein kinase.

The invention provides uses of any (one or more) compound of the invention, or any (one or more) pharmaceutical composition of the invention, or any (one or more) pharmaceutical formulation of the invention, for the manufacture of a medicament for the treatment, prevent or amelioration of diseases or conditions associated with cells activated by a DFG motif-comprising protein kinase.

In alternative embodiments of any use of the invention, the cells are dysfunctional cells, cancer stem cells or cancer cells treated by the medicament are derived from or are cells or stem cells from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma or pituitary adenoma, and any combination thereof.

The invention provides methods for treating, preventing or ameliorating a disease or condition associated with dysfunctional cells, cancer stem cells or cancer cells activated by a DFG motif-comprising protein kinase, comprising

(i) (a) providing any (one or more) compound of the invention, or any (one or more) pharmaceutical composition of the invention, or any (one or more) pharmaceutical formulation of the invention, or any (one or more) liposome, a nanoparticle or a microsphere of the invention; and,

(b) administering to an individual in need thereof an effective amount of the compound, pharmaceutical composition, pharmaceutical formulation or liposome, nanoparticle or microsphere; or

(ii) the method of (i), wherein the individual is a human.

The invention provides methods for treating, preventing or ameliorating a disease or condition associated with cells activated by a DFG motif-comprising protein kinase, comprising

(i) (a) providing any (one or more) compound of the invention, or any (one or more) pharmaceutical composition of the invention, or any (one or more) pharmaceutical formulation of the invention, or any (one or more) liposome, a nanoparticle or a microsphere of the invention; and,

(b) administering to an individual in need thereof an effective amount of the compound, pharmaceutical composition, pharmaceutical formulation or liposome, nanoparticle or microsphere; or

(ii) the method of (i), wherein the individual is a human.

In alternative embodiments of any of the methods of the invention, the cells, dysfunctional cells, cancer stem cells or cancer cells treated by the medicament are derived from or are cells or stem cells from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma or pituitary adenoma, and any combination thereof.

The invention provides methods for inhibiting a DGF motif-comprising protein kinase by disrupting or constraining a kinase-activating molecular rearrangement in the DGF motif, comprising

(i) (a) contacting a protein kinase with any (one or more) compound of the invention, or any (one or more) pharmaceutical composition of the invention, or any (one or more) pharmaceutical formulation of the invention, or any (one or more) liposome, a nanoparticle or a microsphere of the invention; or

(b) contacting a protein kinase-comprising cell with any (one or more) compound of the invention, or any (one or more) pharmaceutical composition of the invention, or any (one or more) pharmaceutical formulation of the invention, or any (one or more) liposome, a nanoparticle or a microsphere of the invention; or

(ii) the method of (i), wherein the contacting is in vitro, ex vivo, or in vivo.

The invention provides methods for inhibiting a kinase-activating molecular rearrangement in a DGF motif (interfering with the activation motion of a DFG switch), comprising:

(a) contacting a kinase with any (one or more) compound of the invention, or any (one or more) pharmaceutical composition of the invention, or any (one or more) pharmaceutical formulation of the invention, or any (one or more) liposome, a nanoparticle or a microsphere of the invention; or

(b) the method of (a), wherein the contacting is in vitro, ex vivo or in vivo.

In alternative embodiments of any of the methods of the invention, the method ameliorates, treats or prevents a disease or condition comprising melanoma and colon cancer angiogenesis; hematopoeitic stem cell function; neurological disorders; cancer; angiogenesis; myeloproliferative disease; diabetes, hypertension, inflammation; Brutons immunodeficiency; neuro-regulator dysfunction, uremia induced hyperplasia, tumorigenesis, invasive metastasis, prostate cancer, breast cancer, colon cancer, pancreatic cancer, a leukemia; thrombosis; abnormal angiogenesis and/or a T cell related disease or condition.

In alternative embodiments of any of the methods of the invention, the kinase is selected from the group consisting of: B-raf, Lsk, c-Src, Fyn, Hck, Epha 3, Eprih A2, Ret, Fak, c-Met, ack1, c-kit, c-Fms, VEGF R2, FGFR1, IL1, EGFR2, Pak1, Ste20, TGFR-beta, c-abl and Tak4.

The invention provides methods for making any compound of the invention, wherein the method comprises the synthetic scheme of FIG. 6.

While the invention is not limited by any particular mechanism of action, compositions of the invention can modulate, e.g., inhibit, the enzymatic activity of a protein kinase by breaking up specific hydrogen bond interactions needed for activation of the kinase's activation domain. The inventors modeled inactive forms of protein kinases and identified specific conserved residues in the activation and phospho-transfer loops required for protein kinase enzyme activity. They then designed inhibitors—compositions of this invention—with near neighbor interactions in these critical regions capable of interfering with these interactions. In alternative embodiments, the resulting novel compounds of this invention are linear molecules within a specific size range and contain heterocyclic ring structures including pyrazolones, imides, pyrolles, imidazoles pyrazoles and triazoles, e.g., asymmetric triazoles. These compounds of this invention represent a new avenue for the treatment development of diseases related to the mis-regulation (dysfunction) of protein kinases.

The inventors discovered that a universally conserved motif in protein kinases, the so-called “DFG motif,” when constrained from undergoing a molecular rearrangement that leads to activation can inhibit enzyme activation, thereby also inhibiting undesirable cellular signals, e.g., that would otherwise lead to or propagate various diseases and conditions. Thus, the invention provides compositions, e.g., small molecules, that can constrain these activating protein kinase “DFG motif” molecular rearrangements, thereby inhibiting and/or abrogating activation or these protein kinases. Additionally, based on examination of protein structures within a confined region that includes the “DFG motif” switch (the activating molecular rearrangement), this invention defines and describes specific molecular parameters for the design of novel inhibitors with unique structural characteristics.

In alternative embodiments, compounds of the invention comprise/consist of (are) compounds having a formula (a) as set forth in FIGS. 3 to 9, wherein the R₁, R₂ and/or the R₃ group are independently selected from the group consisting of hydrogen, halo, hydroxy (—OH), thiol (—SH), cyano (—CN), formyl (—CHO), alkyl, haloalkyl, alkene, alkenyl, alkynyl, heteroalkyl, heteroalkenyl, heteroalkynyl, aryl, substituted aryl, amino, nitro (—NO₂), alkoxy, haloalkoxy, thioalkoxy, alkanoyl, haloalkanoyl and carbonyloxy; (b) the formula of (a), wherein the alkyl, haloalkyl, alkene, alkenyl in both or either of the R₁ and/or the R₂ groups is at least 1, 2, 3, 4, 5, 6, 7, 8, 9, 10, 11, 12, 13, 14, 15 or 16 or more carbons in length; (c) the formula of (a) or (b), wherein the heterocyclic moiety comprises a 5 membered ring or a 6 membered ring system; or (d) the formula of (c), wherein 5 membered ring system comprises an imidazole, thiazole, triazole or oxadiazole, or the 6 membered ring system comprises a pyridine, a pyrimidine or a pyrazine.

In alternative embodiments, compounds of the invention comprise/consist of “alkenyl” and “alkynyl” groups, e.g., which can be similar to alkyl groups, and include straight-chain, branched-chain and cyclic monovalent hydrocarbyl radicals, and combinations of these, which contain only C and H when they are unsubstituted. Alkenyl groups can contain one or more carbon-carbon double bonds, and alkynyl groups can contain one or more carbon-carbon triple bonds.

In alternative embodiments, compounds of the invention comprise/consist of alkyl, alkenyl and alkynyl substituents containing 1-8C (alkyl) or 2-8C (alkenyl or alkynyl). In alternative aspects, they contain 1-4C (alkyl) or 2-4C (alkenyl or alkynyl), and the compound has protein kinase modulating, e.g., inhibiting or diminishing, activity, for example, the composition binds to a protein kinase allosteric binding site involving the DFG motif of the activation loop of the kinase.

In alternative embodiments, compounds of the invention comprise/consist of alkyl, alkenyl and alkynyl groups substituted to the extent that such substitution makes sense chemically and the compound has protein kinase modulating, e.g., inhibiting, activity, for example, the composition binds to a protein kinase in its DFG motif allosteric binding site. In alternative aspects, substituents include, but are not limited to, halo, ═O, —CN, —OR′, —SR′, —S(O)R′, —SO₂R′, —COOR′, —C(O)NR′₂, —NR′₂ and —NHC(═NH)NH₂, where each R′ independently represents H, C1-C4 alkyl or C5-C12 arylalkyl, or a heteroform of one of these, and the compound has protein kinase modulating, e.g., inhibiting, activity, for example, the composition binds to a protein kinase in its DFG motif allosteric binding site.

In alternative embodiments, compounds of the invention comprise “heteroalkyl”, “heteroalkenyl” and/or “heteroalkynyl” groups, and similar groups, which can be similar to the corresponding hydrocarbyl (alkyl, alkenyl and alkynyl) groups, but the ‘hetero’ terms refer to groups that contain one or more heteroatoms selected from O, S and N and combinations thereof, within the backbone residue. In alternative aspects, when heteroatoms, e.g., N, O and S, are allowed to replace carbon atoms of an alkyl, alkenyl or alkynyl group, as in heteroalkyl groups, the numbers describing the group, though still written as e.g. C1-C6, represent the sum of the number of carbon atoms in the group plus the number of such heteroatoms that are included as replacements for carbon atoms in the ring or chain being described. Such heteroalkyl groups may be optionally substituted with the same substituents as alkyl groups.

In alternative aspects, where such groups contain N, the nitrogen atom can be present as an NH moiety, or it may be substituted if the heteroalkyl or similar group is substituted. When such groups contain S, the sulfur atom can be oxidized to SO or SO₂ unless otherwise indicated. For reasons of chemical stability, it is also understood that, unless otherwise specified, such groups do not include more than two contiguous heteroatoms as part of the heteroalkyl chain, although an oxo group may be present on N or S as in a nitro or sulfonyl group. Thus, in alternative aspects, —C(O)NH₂ can be a C2 heteroalkyl group substituted with ═O; and —SO₂NH— can be a C2 heteroalkylene, where S replaces one carbon, N replaces one carbon, and S is substituted with two ═O groups.

While “alkyl” in one aspect includes cycloalkyl and cycloalkylalkyl groups, in other embodiments a composition of the invention can comprise “cycloalkyl” groups, e.g., a carbocyclic non-aromatic group that is connected via a ring carbon atom, or alternatively a “cycloalkylalkyl” of a composition of this invention can be a carbocyclic non-aromatic group that is connected to the base molecule through an alkyl linker. For example, in one embodiment, a composition of the invention can comprise a cyclohexylalanine (Cha) group, and it can comprise a cycloalkylalkyl substituent. In one embodiment, a composition of the invention can comprise a “heterocyclyl”, e.g., a non-aromatic cyclic group that comprises at least one heteroatom as a ring member and that is connected to the molecule via a ring atom of the cyclic group, which may be C or N. In one embodiment, a composition of the invention can comprise a “heterocyclylalkyl”, e.g., a group that is connected to another molecule through an alkyl linker. The sizes and substituents that are suitable for the cycloalkyl, cycloalkylalkyl, heterocyclyl, and heterocyclylalkyl groups in compositions of the invention can be the same as those described above for alkyl groups. In one embodiment, an alkyl group is substituted with an aryl or heteroaryl group, and it can comprise an arylalkyl or heteroarylalkyl substituent.

In one aspect, compositions of the invention comprise one or more “aromatic” moieties or “aryl” moieties, including a monocyclic or fused bicyclic moiety having the well-known characteristics of aromaticity; for example, compositions of the invention can comprise phenyl and naphthyl. Similarly, compositions of the invention can comprise “heteroaromatic” and “heteroaryl” groups such monocyclic or fused bicyclic ring systems, which in alternative embodiments can comprise ring members having one or more heteroatoms, e.g., selected from O, S and N. In these embodiments, the inclusion of a heteroatom permits aromaticity in 5-membered rings as well as 6-membered rings. Heteroaromatic systems used in compositions of this invention include monocyclic C5-C6 aromatic groups such as pyridyl, pyrimidyl, pyrazinyl, pyridazinyl, triazinyl, thienyl, furanyl, pyrrolyl, pyrazolyl, thiazolyl, isothiazolyl, oxazolyl, isoxazolyl, imidazolyl, triazolyl, thiadiazolyl, oxadiazolyl, and tetrazolyl rings. In one aspect, the fused bicyclic moieties are formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolinyl, quinolinyl, benzothiazolyl, benzofuranyl, benzothienyl, benzisoxazolyl, pyrazolopyridyl, quinazolinyl, quinoxalinyl, cinnolinyl, and the like.

Compositions of this invention can comprise any monocyclic or fused ring bicyclic system which has the characteristics of aromaticity in terms of electron distribution throughout the ring system, or can comprise bicyclic groups where at least one ring has the characteristics of aromaticity, even though it may be fused to a nonaromatic ring. Compositions of this invention can comprise ring systems having 5 to 12 ring member atoms. In alternative aspects, the monocyclic heteroaryl groups contain 5 to 6 ring members, and/or the bicyclic heteroaryls can contain 8 to 10 ring members.

Compositions of this invention can comprise aryl and heteroaryl moieties, which in alternative embodiments can be substituted with a variety of substituents which are known in the art. In alternative aspects, such substituents include, but are not limited to, halo, C1-C8 alkyl, —NO₂, —CN, —OR′, —SR′, —COOR′, —C(O)NR′₂, and —NR′₂, where each R′ independently represents H, C1-C4 alkyl or C5-C12 arylalkyl, or a heteroform of one of these.

Similarly, compositions of this invention can comprise “arylalkyl” and “heteroarylalkyl” groups, e.g., aromatic and heteroaromatic ring systems which are bonded to their attachment point through a linking group such as an alkylene, including substituted or unsubstituted, saturated or unsaturated, cyclic or acyclic linkers. Compositions of this invention can comprise a linker, e.g., a C1 to C8 alkyl or a hetero form thereof. Linkers used in compositions of this invention also can include a carbonyl group, thus making them able to provide substituents as an acyl or heteroacyl moieties. Compositions of this invention can comprise a “heteroarylalkyl”, e.g., comprising an aryl group that is attached through a linking group; which can differ from “arylalkyl” in that at least one ring atom of the aryl moiety or one atom in the linking group is a heteroatom selected from N, O and S.

In alternative aspects, an aryl or heteroaryl ring in an arylalkyl or heteroarylalkyl group is substituted on the aromatic portion with the same substituents described above for aryl groups. In alternative embodiments, an arylalkyl group includes a phenyl ring and a heteroarylalkyl group includes a C5-C6 monocyclic or C8-C10 fused bicyclic heteroaromatic ring, each of which can be substituted with the groups defined above for aryl groups and a C1-C4 alkylene that is unsubstituted or is substituted with one or two C1-C4 alkyl groups, where the alkyl groups can optionally cyclize to form a ring, and wherein the alkyl or heteroalkyl groups may be optionally fluorinated. In certain embodiments, the arylalkyl or heteroarylalkyl ring comprises a phenol or an indole ring. In alternative aspects, substituents on phenyl include OH, C1-C4 alkoxy, and halo.

Compositions of this invention can comprise an “arylalkyl” and/or “heteroarylalkyl” group, e.g., as described by the total number of carbon atoms in the ring and alkylene or similar linker. In alternative embodiments, a benzyl group is a C7-arylalkyl group, and phenethyl is a C8-arylalkyl group.

Compositions of this invention can comprise an “alkylene”, which in one aspect refers to a divalent hydrocarbyl group; because it is divalent, it can link two other groups together. Compositions of this invention can comprise —(CH₂)ₙ— where n is 1 to 8, or n is 1 to 4. In alternative embodiments, an alkylene can also be substituted by other groups, and can be of other lengths, and the open valences need not be at opposite ends of a chain. Compositions of this invention can comprise —CH(Me)- and/or —C(Me)₂-, which can be referred to as alkylenes; in alternative embodiments the compositions comprise a cyclic group such as cyclopropan-1,1-diyl. In alternative embodiments, a three-atom linker that is an alkylene group, for example, can be a divalent group in which the available valences for attachment to other groups are separated by three atoms such as —(CH₂)₃—, i.e., the specified length represents the number of atoms linking the attachment points rather than the total number of atoms in the hydrocarbyl group: —C(Me)₂- would thus be a one-atom linker, since the available valences are separated by only one atom. In alternative embodiments, where an alkylene group is substituted, the substituents include those typically present on alkyl groups as described herein, thus —C(═O)— is an example of a one-carbon substituted alkylene. In alternative embodiments, where a compound of the invention is unsaturated, the alkylene may contain one or more double or triple bonds.

Compositions of this invention can comprise a “heteroalkylene”, which in one aspect is similar to the corresponding alkylene groups, and in one aspect the ‘hetero’ terms include groups that contain one or more heteroatoms selected from O, S and N and combinations thereof, within the backbone residue; thus at least one carbon atom of a corresponding alkylene group is replaced by one of the specified heteroatoms to form a heteroalkylene group. Thus, in alternative embodiments —C(═O)NH— is an example of an exemplary two-carbon substituted heteroalkylene composition of this invention, where N replaces one carbon, and C is substituted with a ═O group.

Compositions of this invention can comprise an “aminoalkyl” group, e.g., a C1 to C6 alkyl group that is substituted with at least one amine group having the formula —NR2, where each R is independently H, C1 to C8 alkyl, C5 to C12 aryl and C5 to C12 arylalkyl, or a heteroform of one of these. Compositions of this invention can comprise aminoalkyl groups, which in alternative aspects are substituted on the alkyl portion with one or more other groups suitable as substituents for an alkyl group. In some embodiments, the aminoalkyl substituent is a 1-aminoalkyl group such as a 1-aminomethyl, 1-aminoethyl, 1-aminopropyl or 1-aminobutyl group. In certain embodiments, the aminoalkyl group may comprise a protected amine. One of skill in the art would appreciate that appropriate amine protecting groups may vary depending on the functionality present in the particular monomer. Suitably protected amines may include, for example, carbamates (e.g. tert-butoxycarbonyl, benzyloxycarbonyl, fluorenylmethyloxy-carbonyl, allyloxycarbonyl or (trialkylsilyl)ethoxycarbonyl), carboxamides (e.g. formyl, acyl or trifluoroacetyl), sulfonamides, phthalimides, Schiff base derivatives, and the like. In certain embodiments, an aminoalkyl group may be coupled through an alkylene or heteroalkylene linker to a group such as biotin, or a fluorophore-containing group, such as rhodamine, and such compounds may be useful for screening or mechanistic studies.

Compositions of this invention can comprise a “heteroform”, which can comprise a derivative of a group such as an alkyl, aryl, or acyl, wherein at least one carbon atom of the designated carbocyclic group has been replaced by a heteroatom selected from N, O and S. Thus, in alternative embodiments, the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. In alternative embodiments, no more than two N, O or S atoms are ordinarily connected sequentially, except where an oxo group is attached to N or S to form a nitro or sulfonyl group.

Compositions of this invention can comprise an “optionally substituted” form, which in one aspect indicates that the particular group or groups being described may have no non-hydrogen substituents, or the group or groups may have one or more non-hydrogen substituents. In alternative embodiments, if not otherwise specified, the total number of such substituents that may be present is equal to the number of H atoms present on the unsubstituted form of the group being described. In alternative embodiments, where an optional substituent is attached via a double bond, such as a carbonyl oxygen (═O), the group takes up two available valences, so the total number of substituents that may be included is reduced according to the number of available valences.

Compositions of this invention can comprise a “halo” group, which in one aspect includes fluoro, chloro, bromo and iodo. Fluoro and chloro also can be used.

Compositions of this invention can comprise an “amino” group, which in one aspect comprises NR′₂ wherein each R′ is independently H, or is an alkyl, alkenyl, alkynyl, acyl, aryl, or arylalkyl group or a heteroform of one of these groups, as defined above; and in alternative embodiments each of these is substituted with the substituents described herein as suitable for the corresponding type of group. In certain embodiments, the two R′ groups on one nitrogen atom may be linked together to form an azacyclic ring.

Compositions of this invention can comprise an ‘azacyclic’ group, which in one aspect is a heterocyclic group containing at least one nitrogen atom as a ring atom, wherein the group is attached to the base molecule through a nitrogen atom of the azacyclic group. Compositions of this invention can comprise azacyclic groups, e.g., as 3 to 8 membered monocyclic rings or 8 to 12 membered bicyclic fused ring systems, and in alternative aspects are saturated, unsaturated and/or aromatic and may contain a total of 1 to 3 heteroatoms independently selected from N, O and S as ring members. In certain embodiments, an azacyclic ring comprises a nitrogen-containing ring fused to a phenyl ring. For example, in alternative embodiments compositions of this invention comprise the unnatural amino acid “Tic” comprising a tetrahydroisoquinoline ring, which can represent a 10-membered fused bicyclic azacyclic group.

In alternative embodiments, the compositions of the invention, including the DFG motif-inhibiting compounds of the invention; and analogs thereof, which are also compositions of this invention, and pharmaceutical compositions and formulations comprising them, and methods of the invention, are used to treat, prevent or ameliorate (including slowing the progression of) dysfunctional (e.g., abnormally proliferating, differentiating or de-differentiating) cells, including pluripotent cells or stem cells, and cancer or tumor stem cells, and/or to treat, prevent or ameliorate abnormally activated cells causing or involved in, e.g., metabolic, bone, muscle, vasculature or neurological conditions or disorders.

In alternative embodiments, the invention provides methods for modulating, e.g., inhibiting, an enzyme, e.g., a protein kinase activity; which in one embodiment comprises contacting a DFG motif-comprising enzyme (e.g., a protein kinase) with any compound of the invention, or the pharmaceutical composition of the invention, or the liposome of the invention, or the nanoparticle of the invention; wherein in alternative aspects the contacting is in vitro, ex vivo or in vivo.

In alternative embodiments, the compounds of the invention, which includes analogs of compositions of the invention, and the formulations and pharmaceutical compositions comprising them, and methods, are used to stop, reverse or slow the growth and/or proliferation of dysfunctional (e.g., abnormally proliferating, differentiating or de-differentiating) cells, including pluripotent cells or stem cells, or cancer or tumor stem cells, or any cells causing or involved in, e.g., metabolic, bone, muscle, vasculature or neurological conditions or disorders.

The compositions and methods of the invention, including the formulations and pharmaceutical compositions of the invention, can be useful to treat, reverse, prevent (prophylaxis) or ameliorate any dysfunctional cell, or any abnormally (dysfunctional) dividing or metastasizing cell, e.g., any cancer stem cell or a tumor stem cell. Cancers that can be treated, prevented or ameliorated by using compositions and methods of this invention include lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and/or any combination thereof.

Also provided herein are kits comprising the compositions and methods of this invention, and instructions for making, formulating and/or using them, e.g., for the therapeutic and/or prophylactic applications as described herein.

The invention provides liposomes, microspheres, nanoparticles and the like, comprising any compound of this invention, or a compound made by any of the biosynthetic or synthetic methods of this invention.

The invention provides uses of compounds of this invention for the manufacture of a medicament, e.g., for the treatment, prevent or amelioration of diseases or conditions associated with dysfunctional cells, including any abnormally proliferating, differentiating or de-differentiating cell, also including cancer cells and cancer stem cells, including cancer cells and cancer stem cells associated with lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma or pituitary adenoma, and any combination thereof.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 illustrates the location of an Asp-Phe-Gly, or “DFG”, activity on-off switch in the active form of a protein kinase, as discussed in detail, below. FIG. 1A illustrates the glycine of DFG moving out of its amide bond upon inactivation, breaking an H-bond; FIG. 1B illustrates alignment of the magnesium (Mg) binding loop of 23 active kinases, and arrows show conserved H-bond between DG and DFG+2; and, FIG. 1C illustrates formation of b6/b9 sheets conserved H-bonds.

FIG. 2 illustrates the inactive form configuration of a kinase enzyme activation loop, which includes a DFG switch, as discussed in detail, below.

FIG. 3 illustrates superimposed crystal structures of kinases inhibited by compounds of this invention, including B-raf, C-src, Fak, Pak-1, C-abl, C-met, C-kit, VEGF-R, PDGF-R and C-FMS, as discussed in detail, below.

FIG. 4A illustrates a protein kinase, and specifically, it illustrates the location of kinase substructures, including inhibitor binding sites, within the kinase domain as exemplified for the kinase c-FMS that can acts as targets for compounds of this invention, as discussed in detail, below. FIG. 4B1 and FIG. 4B2 illustrate exemplary compounds of this invention that bind to, e.g., the illustrated kinase c-FMS, as discussed in detail, below.

FIG. 5 illustrates exemplary heterocycles contained within (as part of the structure of) compounds of this invention, as discussed in detail, below.

FIG. 6 illustrates an exemplary synthetic scheme for making compounds of this invention, as discussed in detail, below.

FIG. 7A illustrates docking (to a protein kinase) of exemplary compounds of the invention with the asymmetric triazole ring, as discussed in detail, below. FIG. 7B illustrates exemplary compounds of the invention for inhibiting kinase activation, e.g., c-kit activation.

FIG. 8 illustrates a computer model of an exemplary composition of this invention, an oxadiazole-amide inhibitor structure, binding to a c-kit kinase allosteric binding domain, as discussed in detail, below.

FIG. 9 illustrates Table 2, an illustration of the rational for an exemplary protocol of this invention for drug targeting of A-loops of inactive kinases, highlighting indicate mutations in kinases found in cancer patients, as discussed in detail, below.

Like reference symbols in the various drawings indicate like elements.

### DETAILED DESCRIPTION

The invention provides compositions, e.g., small molecules, that disrupt (e.g., inhibit or diminish) the activity of protein kinases, including constitutively activated protein kinases, such as kinases constitutively activated through mutations, and kinases in their native inactivated state, and methods for making and using them. In one aspect, compositions of the invention bind a protein kinase in its inactive conformation involving the conserved asparagine-phenylalanine-glycine residue motif, or “Asp-Phe-Gly” or “DFG” motif, of the activation loop in the allosteric binding site. In one aspect, when a composition of this invention binds to a protein kinase in its inactive conformation, this binding inhibits or diminishes the activation of the kinase, and in one aspect, this activation-inhibiting binding is reversible, or alternatively the activation-inhibiting binding can be irreversible. In one aspect, when a composition of this invention binds to a protein kinase constitutively activated through mutations, this binding inhibits or diminishes (reversibly or irreversibly) the activity of that constitutively active kinase.

The small molecule protein kinase inhibitors of the invention comprise (have the structure of), or are derivatives or analogs of, oxadiazoles, thiadiazoles, oxazoles, thiazoles, arylamides, quinolones, pyrazoles, pyrazolones, imides, pyrolles, imidazoles and/or triazoles, as described herein.

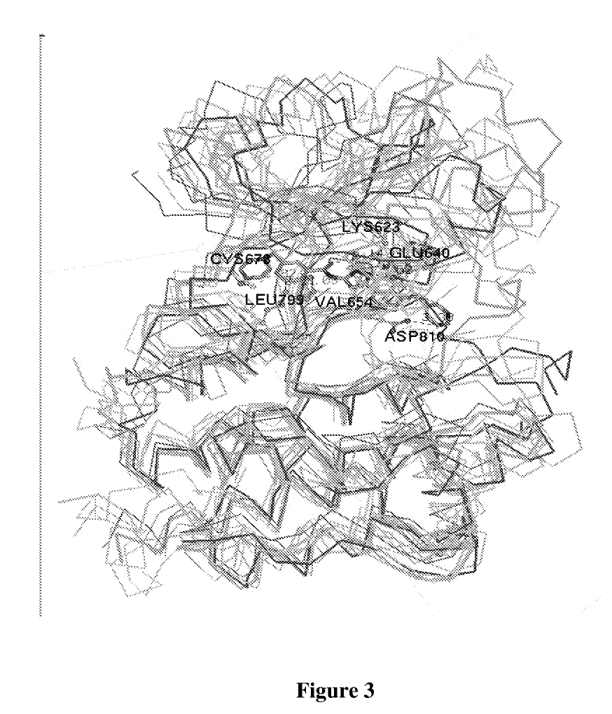

In alternative embodiments, the invention provides compositions comprising any of the compounds illustrated in FIGS. 3 to 9, or as described or illustrated herein, and analogs thereof, and formulations and pharmaceutical compositions comprising them, and methods for inhibiting, ameliorating or preventing cancer (tumor) cell growth. In alternative embodiments, the compositions and methods of the invention, and the pharmaceutical compositions comprising them, are used to treat, prevent or ameliorate (including slowing the progression of) a cancer, or a vasculature or neurological disorder.

Compositions of the invention can be used to inhibit, abrogate or reverse the abnormal signaling of protein kinases (including enzymes abnormally activated either through mutations, phosphorylation, or protein-protein interactions), thereby inhibiting, ameliorating or preventing cellular aberrations associated with a variety of disease states, e.g., cancer or diseases of the vasculature or neurological disorders. Thus, the compositions of this invention can be used as anticancer drugs, anti-inflammatory drugs, drugs to treat neurological disorders and/or drugs to treat disorders of the vascular system, and prophylactic uses thereof.

The invention provides compounds which bind in the allosteric binding site of DFG motif-comprising protein kinases, e.g., bRaf. In one aspect, this invention uses heterocyclic rings and/or varying heterocyclic structures to disrupt specific hydrogen bond interactions in the DFG motif. Thus, the compositions of the invention can specifically bind to the DFG region, thereby modulating kinase activity with specificity.

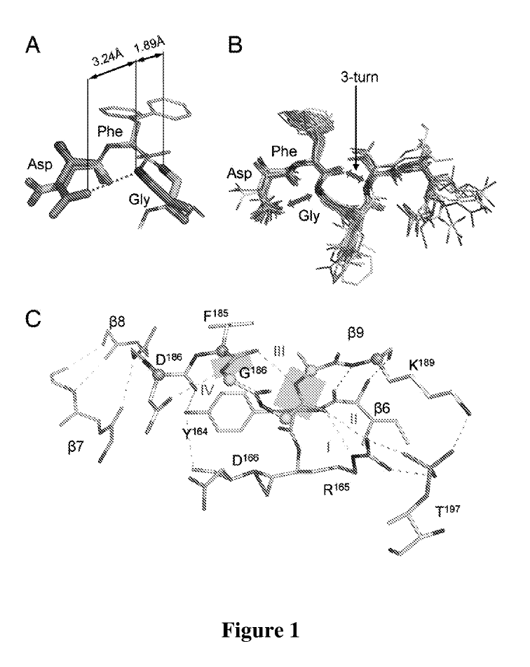

The location of DFG switch in the active forms of protein kinases is similar for all protein kinases, see FIG. 1, which is an illustration of a kinase “DFG switch” mechanism, where FIG. 1A illustrates the glycine of DFG moving out of its amide bond upon inactivation, breaking an H-bond; FIG. 1B illustrates alignment of magnesium (Mg) binding loop of 23 active kinases, and arrows show conserved H-bond between DG and DFG+2; and, FIG. 1C illustrates formation of b6/b9 sheets conserved H-bonds.

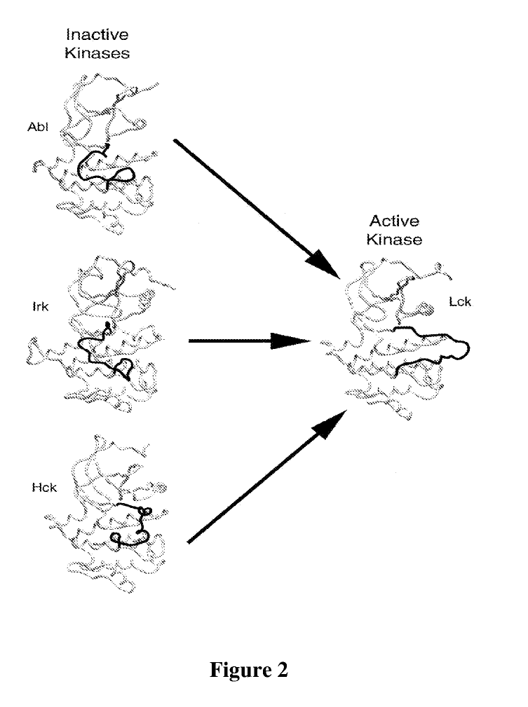

However, in the inactive form the configuration of the activation loop, which includes this DFG switch, is shifted to different locations, see FIG. 2, which (although the invention is not limited by any particular mechanism of action) illustrates an exemplary rational for targeting inactive kinase conformations, also illustrating the selectivity of the compounds of the invention towards kinases.

In one embodiment, this invention takes advantage of these differences in location (configuration of the activation loop) to design and confirm the activity of blocking molecules of this invention, e.g., to confirm that, in one aspect (although the invention is not limited by any particular mechanism of action—e.g., not necessarily limited to any particular binding site on a kinase to inhibit the kinase) compositions of this invention can prevent this “switch” in the activation loop from moving (re-configuring) into an activation mode. The crystal structures of many protein kinases in the inactive forms are known (have been published (see e.g., Table 1, below), thus protein kinase crystal structures can be used to design specific molecules to restrain the movement of a DFG triad into the “on” position (active) from an inactive configuration; or to confirm in silico the kinase-binding and kinase-inhibiting activity of compositions of the invention.

Mis-regulated and/or mutated protein kinases whose activation can be modulated, e.g., inhibited, by a compound of this invention include: B-raf, to treat, prevent or ameliorate, e.g., melanoma and colon cancer angiogenesis; Lsk, to treat, prevent or ameliorate, e.g., hematopoeitic stem cell function; c-Src, to treat, prevent or ameliorate, e.g., cancer, angiogenesis; Fyn, to treat, prevent or ameliorate, e.g., oncogenic, neurologic disorders; Hck, to treat, prevent or ameliorate, e.g., myeloproliferative disease; Epha 3, to treat, prevent or ameliorate, e.g., neurological disorders; Eprih A2, to treat, prevent or ameliorate, e.g., diabetes, hypertension and/or inflammation; Brutons, to treat, prevent or ameliorate, e.g., immunodeficiency; Ret, to treat, prevent or ameliorate, e.g., neuroregulator function; Fak, to treat, prevent or ameliorate, e.g., uremia induced hyperplasia; c-Met, to treat, prevent or ameliorate, e.g., tumorigenesis, invasive metastasis; ack1, to treat, prevent or ameliorate, e.g., promotes prostate cancer progression; c-kit, to treat, prevent or ameliorate, e.g., colon cancer, leukemias; c-Fms, to treat, prevent or ameliorate, e.g., thrombosis, cancer; VEGF R2, to treat, prevent or ameliorate, e.g., angiogenesis, apoptosis; FGFR1, to treat, prevent or ameliorate, e.g., vasular associated diseases; IL1 to treat, prevent or ameliorate, e.g., an inflammatory disease; EGFR2, to treat, prevent or ameliorate, e.g., a cancer; Pak1, to treat, prevent or ameliorate, e.g., angiogenesis and/or cancer; Ste20, to treat, prevent or ameliorate, e.g., colorectal cancer; TGFR-beta, to treat, prevent or ameliorate, e.g., T cell related diseases; IGF, to treat, prevent or ameliorate, e.g., breast cancer; c-abl, to treat, prevent or ameliorate, e.g., a leukemia, CML; PDGF-R, to treat, prevent or ameliorate, e.g., angiogenesis and/or cancer; and/or Tak4, to treat, prevent or ameliorate, e.g., pancreatic tumor.

TABLE 1
Table 1. Crystal Structures of Inactive Protein Kinases

| | | | Reso- | | |
|---|---|---|---|---|---|
| | | | lution | | |
| Kinase | Group | PDB | (A) | R [symbol] (%) | Reference |
| AuroraA | AGC | 1MUO | 2.6 | 26.0 | Cheetham, [symbol] |
| GRK2 | AGC | 1OMW | 2.5 | 25.2 | Lodowski, [symbol] |
| PK [symbol] | AGC | 1MRY | 2.8 | 27.2 | Huang, [symbol] |
| CCK2 | CMGC | 1HCK | 1.9 | 27.2 | Schulze-Gahmen, [symbol] |
| [symbol] RK2 | CMGC | 1GFK | 2.3 | 26.2 | Zhang, [symbol] |
| GSK- [symbol] | CMGC | 1H8F | [symbol] | [symbol] | [symbol] , [symbol] |
| Jnk1 | CMGC | 1UKH | 2.4 | 24.5 | Heo, 2004 |
| Jnk2 | CMGC | [symbol] | 2.3 | [symbol] | [symbol] , [symbol] |
| [symbol] | CMGC | [symbol] | 2.1 | [symbol] | Wang, [symbol] |
| [symbol] | CAMK | [symbol] | 3.5 | 31.3 | Goldberg, [symbol] |
| [symbol] | CAMK | 1TKI | 2.0 | 24.8 | Mayans, [symbol] |
| [symbol] | CAMK | 1KOB | 2.3 | NR [symbol] | Kobe, [symbol] |
| MAPKAPK2 | CAMK | 1NY [symbol] | 2.1 | 28.2 | Underwood, 2003 |
| C-RAF | TKL | 1UWH | 2. [symbol] | [symbol] | [symbol] , [symbol] |
| TGF [symbol] R1 | TKL | [symbol] | 2. [symbol] | [symbol] | [symbol] , 2004 |
| [symbol] TK | TK | 1K [symbol] | 2.1 | [symbol] | [symbol] , [symbol] |
| c-Abl | TK | 1OFJ | 1.7 | 24.2 | Hagar, [symbol] |
| c-KIT | TK | 1T4K | 1.0 | 32.0 | [symbol] , 2004 |
| CSK | TK | 1 [symbol] YG | 3.4 | 28.7 | Lamars, [symbol] |
| c-Src | TK | 1FMK | 1.3 | 28.4 | Xu, [symbol] |
| [symbol] | TK | [symbol] | 2.3 | [symbol] | [symbol] , 2002 |
| [symbol] | TK | [symbol] | 1.0 | [symbol] | [symbol] , 2001 |
| FGFR [symbol] | TK | 1FGN | 3.2 | [symbol] | [symbol] , [symbol] |
| [symbol] | TK | [symbol] | 3.1 | 24.2 | Griffin, 2004 |
| [symbol] | TK | [symbol] | 2.3 | 31.2 | [symbol] , [symbol] |
| IGFIFK | TK | 1MTN | 2.7 | [symbol] | [symbol] , 2003 |
| Irk [symbol] | TK | 1IRK | 3.1 | 23.2 | Hubbard, [symbol] |
| [symbol] 2 | TK | 1FYR | 2.3 | 32.0 | [symbol] , [symbol] |
| [symbol] | TK | 1LUF | 2.1 | [symbol] | [symbol] , 2002 |
| FAK | TK | 1NF [symbol] | 1.7 | 23.6 | [symbol] , 2003 |
| [symbol] | TK | 1RIW | 1.9 | 26.4 | [symbol] , 2003 |
| [symbol] | STE | 1F [symbol] | 2.3 | [symbol] | [symbol] , [symbol] |

[symbol]

In many cases multiple inactive structures have been reported. [symbol] only one [symbol] [symbol] for each inactive kinase in this table. [symbol]
R [symbol] not reported.
GSM- [symbol] lacking [symbol] phosphorylation at 5-fold [symbol] active than phosphorylated GSM- [symbol] [symbol]
indicates data missing or illegible when filed

The inventors performed computer simulations and computations to design the compounds of this invention, which can interfere with the activation motion of the DFG switch. Superposition of exemplary protein kinases inhibited by compounds of this invention are shown in FIG. 3. Overall the structures of these kinases are very similar, providing structural validation for the ability of compounds of this invention to inhibit a broad range of kinase enzymes having the DFG switch motif, including B-raf, Lsk, c-Src, Fyn, Hck, Epha 3, Eprih A2, Ret, Fak, c-Met, ack1, c-kit, c-Fms, VEGF R2, FGFR1, IL1, EGFR2, Pak1, Ste20, TGFR-beta, c-abl and Tak4. Because overall the structures of these kinases are very similar, the common structure of the DFG switch mirrors the structures of inhibitor compounds of this invention to fit into the protein kinase allosteric binding site.

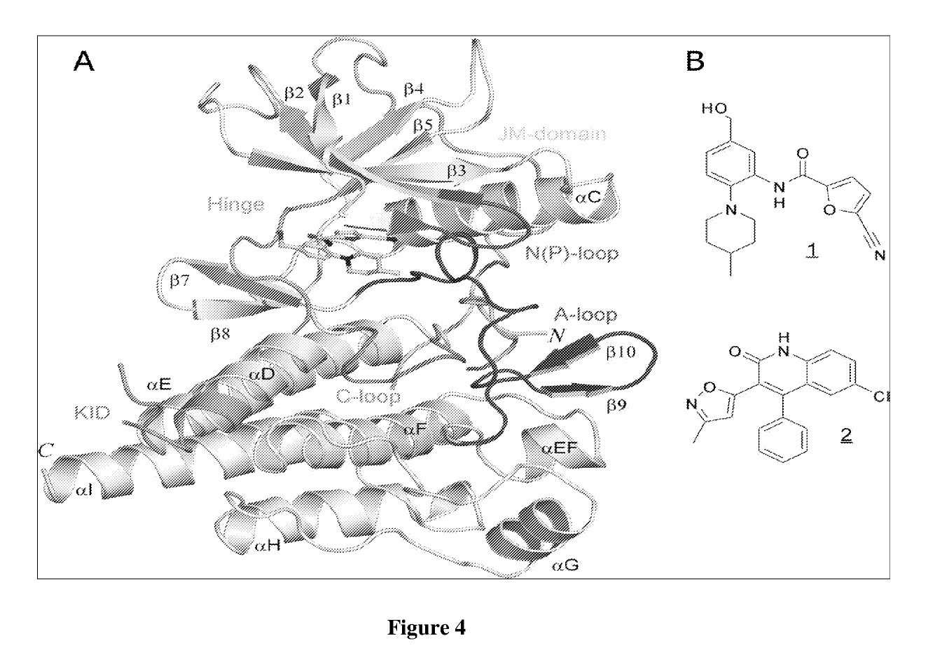

FIG. 4A illustrates a protein kinase, and specifically, it illustrates the location of kinase substructures, including inhibitor binding sites, within the kinase domain as exemplified for the kinase c-FMS. In the illustration of FIG. 4A, the Mg binding loops that include the DFG motif are targets for the structure based designs of this invention, i.e., are targets for compounds of this invention. FIG. 4B1 and FIG. 4B2 illustrate exemplary compounds of this invention that bind to, e.g., the illustrated kinase c-FMS.

While the invention is not limited by any particular mechanism of action, one rational for drug targeting this region is that for kinases of interest in cancer, hot spot mutations are located precisely at the DFG+3 residue of the activation loop; see Table 2, illustrated in FIG. 9, which illustrates an exemplary rational for drug targeting of A-loops of inactive kinases, highlighting indicate mutations in kinases found in cancer patients.

A method of the invention for determining whether a compound binds to a kinase is also illustrated below as a “specific drug design parameter” list and protocol; and exemplary structural genus of this invention is also illustrated.

### Specific Drug Design Parameters

Goal: Targeting Inactive Form of Kinases with DFG Motifs
1. linear conformational restricted chain molecule <12 A length
2. one polar and one apolar end
3. polar end is a five or six membered heterocycle
4. minimum of 4 rings connected by polar linkers x, y and z
5. X is neutral:oxygen, sulfide, sulfoxide or sulfone
6. Y is a polar group, can be H bond donor, or nil
7. Z is a polar H-bond donating group or nil if Y is not nil
8. nonpolar end is a cyclic hydrocarbon: aromatic or alicyclic.
9. two central aromatic rings should be coplanar within 15-20 degrees

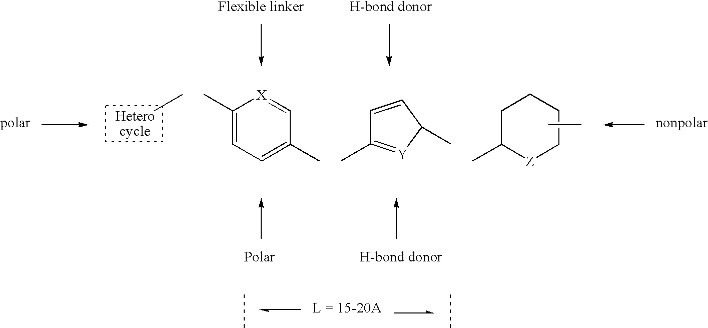

The inventors have shown for the case of b-Raf kinase that disruption of critical amino acid residues within the activation loop of the enzyme near the DFG motif by an exemplary kinase inhibitor of this invention results in biologically active compounds both in vitro and in vivo. The invention also includes compounds whose structures can be manipulated via heterocyclic diversity inputs to block the action of any DFG-comprising protein kinase. Molecules of the invention can be designed and manufacture based on specific drug design parameters, e.g., as outlined in Table 3.

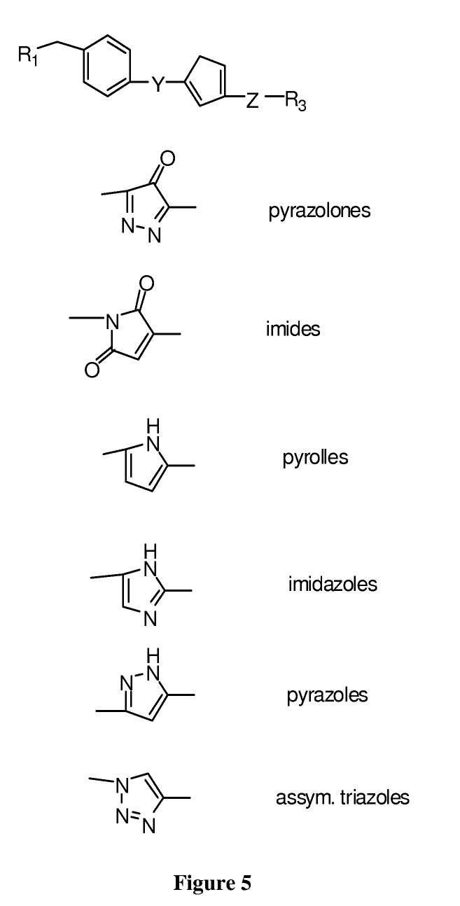

The structures of this invention as shown in FIG. 5, which illustrates exemplary heterocycles contained within (as part of the structure of) compounds of this invention. The structures of this invention as shown in FIG. 5 are used as diversity inputs from which alternative compounds of this invention are synthesized, e.g., with R₁ and R₃ as structural modifications as specified in Table 3.

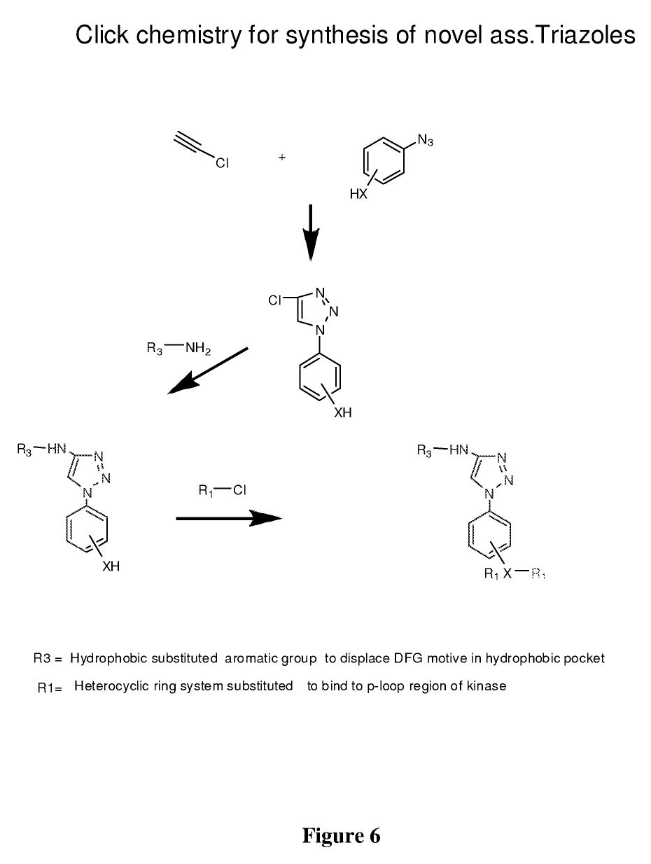

An exemplary synthetic scheme of this invention for exemplary compounds of the invention is illustrated in FIG. 6; note R3 is a hydrophobic substituted aromatic group to prevent the phenyl group of the phenylalanine residue in the DFG motif to move into the hydrophobic pocket and, R1 is a heterocyclic ring system substituted to bind to the kinases' in the hinge region.

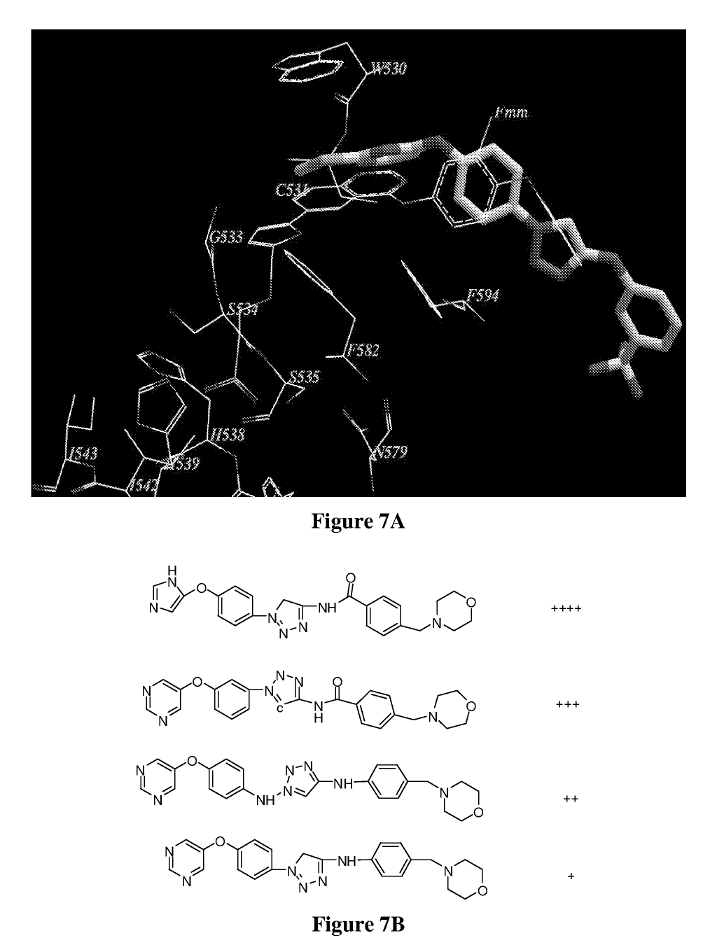

Docking to B-raf kinase of exemplary compounds of the invention having asymmetric triazole rings is illustrated in FIG. 7A; the compounds of the invention are binding to the allosteric site of B-raf; and amino acid residues relevant to the binding of this exemplary composition to the B-raf kinase allosteric binding domain are noted. FIG. 7B illustrates exemplary compounds of the invention for inhibiting kinase activation, e.g., c-kit activation, and designates the strength of binding to the allosteric site of B-raf, where “++++” has (relatively) the strongest binding affinity, and “+” the least (relatively).

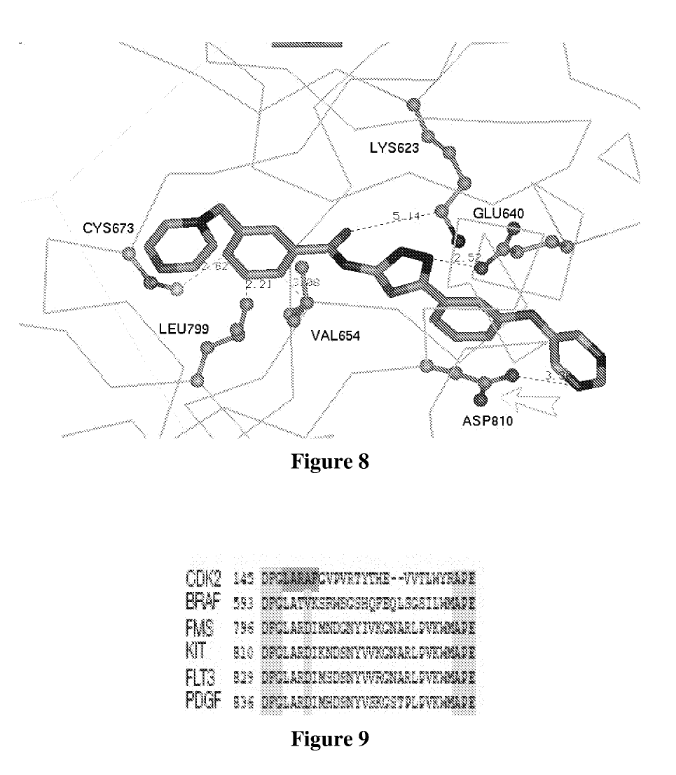

FIG. 8 illustrates a computer model of an exemplary composition of this invention, an oxadiazole-amide inhibitor structure, binding to a c-kit kinase allosteric binding domain. The amino acid residues relevant to the binding of this exemplary composition to the c-kit kinase allosteric binding domain are noted: LYS623, GLU640, ASP810, VAL654, LEU799, CYS673.

### Pharmaceutical Compositions

The invention provides compositions for inhibiting protein kinases, as described herein, including pharmaceutical compositions, e.g., in the manufacture of medicaments for inhibiting the growth of non-normal cells, e.g., cancer cells.

In alternative embodiments, the compositions and analogs of the invention are formulated with a pharmaceutically acceptable carrier. In alternative embodiments, the pharmaceutical compositions of the invention can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Therapeutic agents of the invention can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions and analogs of the invention include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Pharmaceutical formulations of this invention can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., a chimeric polypeptide or peptidomimetic of the invention) in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals are particularly useful for administration of the hydrophobic active agents of the invention, including and analog compositions of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In practicing this invention, the pharmaceutical compounds can also be administered by in intranasal, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In practicing this invention, the pharmaceutical compounds can be delivered by transdermally, by a topical route, formulated as applicator sticks, solutions, suspensions, emulsions, gels, creams, ointments, pastes, jellies, paints, powders, and aerosols.

In practicing this invention, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In practicing this invention, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In alternative embodiments, compounds of the invention are also formulated using cyclodextrins or cycloamyloses, e.g., to take advantage of the ability of cyclodextrins to form complexes with hydrophobic molecules (e.g., compounds of the invention). Mechanically-interlocked molecules structures and architectures, such as rotaxanes and catenanes, can be made using compounds of the invention and cyclodextrins. Cyclodextrins used in these embodiments can be any cyclic oligosaccharide, e.g., composed of 5 or more α-D-glucopyranoside units linked 1->4, as in amylose, a fragment of starch, or, a cyclodextrins comprising glucose monomers ranging from six to eight units in a ring, creating a cone shape α-cyclodextrin: six membered sugar ring molecule, or β-cyclodextrin: seven sugar ring molecule, or γ-cyclodextrin: eight sugar ring molecule. Other cyclodextrins or cycloamyloses that can be used in formulations of this invention are described in, e.g., U.S. Pat. App. Pub. Nos. 20080119431 (describing Per-6-guanidino-, alkylamino-cyclodextrins); 20080091006 (describing nitrate ester cyclodextrin complexes); 20080058427 (describing water-soluble, cyclodextrin-containing polymers with a linear polymer chain for drug delivery); 20070259931; 20070232567; 20070232566; and see also U.S. Pat. No. 7,307,176 (describing a 2-hydroxypropyl-beta-cyclodextrin drug inclusion complex); U.S. Pat. No. 7,270,808 (describing cyclodextrin-containing polymers improve drug stability and solubility, and reduce toxicity of a small molecule therapeutic when used in vivo); U.S. Pat. Nos. 7,262,165; 7,259,153; 7,235,186; 7,157,446; 7,141,555.

The pharmaceutical compounds and formulations of the invention can be lyophilized. The invention provides a stable lyophilized formulation comprising a composition of the invention, which can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations of the invention can be delivered by the use of liposomes (see also discussion, below). By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

The formulations of the invention can be administered for prophylactic and/or therapeutic treatments. In therapeutic applications, compositions are administered to a subject already suffering from a condition, infection or disease in an amount sufficient to cure, alleviate or partially arrest the clinical manifestations of the condition, infection or disease and its complications (a “therapeutically effective amount”). For example, in alternative embodiments, pharmaceutical compositions of the invention are administered in an amount sufficient to treat, prevent and/or ameliorate normal, dysfunction (e.g., abnormally proliferating) blood vessels, including endothelial and/or capillary cell growth; including neovasculature related to (within, providing a blood supply to) hyperplastic tissue, a granuloma or a tumor. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, an exemplary pharmaceutical formulation for oral administration of and analog compositions of the invention in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods of the invention can further comprise co-administration with other drugs or pharmaceuticals, e.g., compositions for treating cancer, septic shock, infection, fever, pain and related symptoms or conditions. For example, the methods and/or compositions and formulations of the invention can be co-formulated with and/or co-administered with antibiotics (e.g., antibacterial or bacteriostatic peptides or proteins), particularly those effective against gram negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

### Nanoparticles and Liposomes

The invention also provides nanoparticles and liposomal membranes comprising compounds of this invention which target specific molecules, including biologic molecules, such as polypeptide, including cell surface polypeptides. Thus, in alternative embodiments, the invention provides nanoparticles and liposomal membranes targeting diseased and/or tumor (cancer) cells, dysfunctional stem cells and any dysfunctional cell.

In alternative embodiments, the invention provides nanoparticles and liposomal membranes comprising (in addition to comprising compounds of this invention) molecules, e.g., peptides or antibodies, that selectively target diseased, infected, dysfunctional and/or cancer (tumor) cell receptors. In alternative embodiments, the invention provides nanoparticles and liposomal membranes using IL-11 receptor and/or the GRP78 receptor to targeted receptors on cells, e.g., on tumor cells, e.g., on prostate or ovarian cancer cells. See, e.g., U.S. patent application publication no. 20060239968.

Thus, in one aspect, the compositions of the invention are specifically targeted for inhibiting, ameliorating and/or preventing endothelial cell migration and for inhibiting angiogenesis, e.g., tumor-associated or disease- or infection-associated neovasculature.

The invention also provides nanocells to allow the sequential delivery of two different therapeutic agents with different modes of action or different pharmacokinetics, at least one of which comprises a composition of this invention. A nanocell is formed by encapsulating a nanocore with a first agent inside a lipid vesicle containing a second agent; see, e.g., Sengupta, et al., U.S. Pat. Pub. No. 20050266067. The agent in the outer lipid compartment is released first and may exert its effect before the agent in the nanocore is released. The nanocell delivery system may be formulated in any pharmaceutical composition for delivery to patients suffering from a diseases or condition as described herein, e.g., such as cancer, inflammatory diseases such as asthma, autoimmune diseases such as rheumatoid arthritis or infectious diseases. In treating cancer, a traditional antineoplastic agent is contained in the outer lipid vesicle of the nanocell, and an antiangiogenic agent of this invention is loaded into the nanocore. This arrangement allows the antineoplastic agent to be released first and delivered to the tumor before the tumor's blood supply is cut off by the composition of this invention.

The invention also provides multilayered liposomes comprising compounds of this invention, e.g., for transdermal absorption, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition of this invention.

A multilayered liposome of the invention may further include an antiseptic, an antioxidant, a stabilizer, a thickener, and the like to improve stability. Synthetic and natural antiseptics can be used, e.g., in an amount of 0.01% to 20%. Antioxidants can be used, e.g., BHT, erysorbate, tocopherol, astaxanthin, vegetable flavonoid, and derivatives thereof, or a plant-derived antioxidizing substance. A stabilizer can be used to stabilize liposome structure, e.g., polyols and sugars. Exemplary polyols include butylene glycol, polyethylene glycol, propylene glycol, dipropylene glycol and ethyl carbitol; examples of sugars are trehalose, sucrose, mannitol, sorbitol and chitosan, or a monosaccharides or an oligosaccharides, or a high molecular weight starch. A thickener can be used for improving the dispersion stability of constructed liposomes in water, e.g., a natural thickener or an acrylamide, or a synthetic polymeric thickener. Exemplary thickeners include natural polymers, such as acacia gum, xanthan gum, gellan gum, locust bean gum and starch, cellulose derivatives, such as hydroxy ethylcellulose, hydroxypropyl cellulose and carboxymethyl cellulose, synthetic polymers, such as polyacrylic acid, poly-acrylamide or polyvinylpyrollidone and polyvinylalcohol, and copolymers thereof or cross-linked materials.

Liposomes can be made using any method, e.g., as described in Park, et al., U.S. Pat. Pub. No. 20070042031, including method of producing a liposome by encapsulating a therapeutic product comprising providing an aqueous solution in a first reservoir; providing an organic lipid solution in a second reservoir, wherein one of the aqueous solution and the organic lipid solution includes a therapeutic product; mixing the aqueous solution with said organic lipid solution in a first mixing region to produce a liposome solution, wherein the organic lipid solution mixes with said aqueous solution so as to substantially instantaneously produce a liposome encapsulating the therapeutic product; and immediately thereafter mixing the liposome solution with a buffer solution to produce a diluted liposome solution.

The invention also provides nanoparticles comprising compounds of this invention to deliver a composition of the invention as a drug-containing nanoparticles (e.g., a secondary nanoparticle), as described, e.g., in U.S. Pat. Pub. No. 20070077286. In one embodiment, the invention provides nanoparticles comprising a fat-soluble drug of this invention or a fat-solubilized water-soluble drug to act with a bivalent or trivalent metal salt.

### Kits and Libraries

The invention provides kits comprising compositions of this invention and methods of the invention, including cells and/or fish of the invention, target sequences, transfecting agents, transducing agents, instructions (regarding the methods of the invention), or any combination thereof. As such, kits, cells, vectors and the like are provided herein.

While the invention has been described in detail with reference to certain exemplary aspects thereof, it will be understood that modifications and variations are within the spirit and scope of that which is described and claimed.

A number of aspects of the invention have been described. Nevertheless, it will be understood that various modifications may be made without departing from the spirit and scope of the invention. Accordingly, other aspects are within the scope of the following claims.

1. A compound having formula (I) 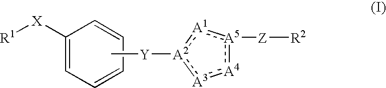
and pharmaceutically acceptable salts thereof,
wherein R¹ is an optionally substituted 5-6 membered heterocyclic ring;
R² is a 3-7 membered alicyclic ring or a 5-6 membered aromatic ring, each of which may be optionally substituted;
X is CH₂, O, S, SO, SO₂, NH or C═O;
Y is a bond or NH, O or S;
Z is a bond, NH, O, S, NH(C═O), or NHSO₂;
A¹ is CH, NH, CH₂, C═O, O or S; and
A², A³, A⁴, and A⁵ are independently C, CH, NH or N.

2. The compound of claim 1, wherein:
(a) R¹ is selected from the group consisting of
pyridinyl, pyrazinyl, pyrimidinyl, pyridazinyl, triazinyl,
pyrrolyl, pyrazolyl, imidazolyl, triazolyl, pyrazolinyl,
piperidinyl, piperazinyl, pyrrolidinyl,
furanyl, pyranyl, tetrahydrofuranyl, dioxanyl, thiophenyl,
oxazolyl, isoxazolyl, oxadiazolyl, thiazolyl, isothiazolyl, thiadiazolyl,
morpholinyl, and 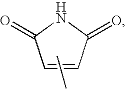
each of which is optionally substituted with one or more substituents;
(b) R² is a 5-6 membered aromatic ring wherein the aromatic ring is phenyl, pyrrolyl, thiophenyl, furanyl, pyridinyl or pyranyl;
(c) A², A³ and A⁴ are N;
(d) A¹ is CH and A⁵ is C;
(e) the compound is comprised of at least five rings;
(f) R² is selected from the group consisting of phenyl, cyclopentadiene, and C3-C6 cycloalkyl, each of which is optionally substituted; or
(g) R² is phenyl, wherein phenyl is substituted with

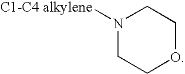

3-8. (canceled)

9. The compound of claim 1, wherein the formula depicted as 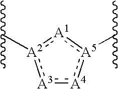
is selected from a group consisting of

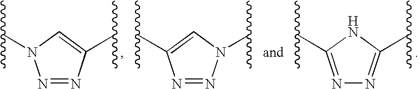

10. The compound of claim 1, wherein
(a) X is O; or
(b) R¹ is imidazolyl or pyrimidinyl.

11. (canceled)

12. A compound from the group consisting of

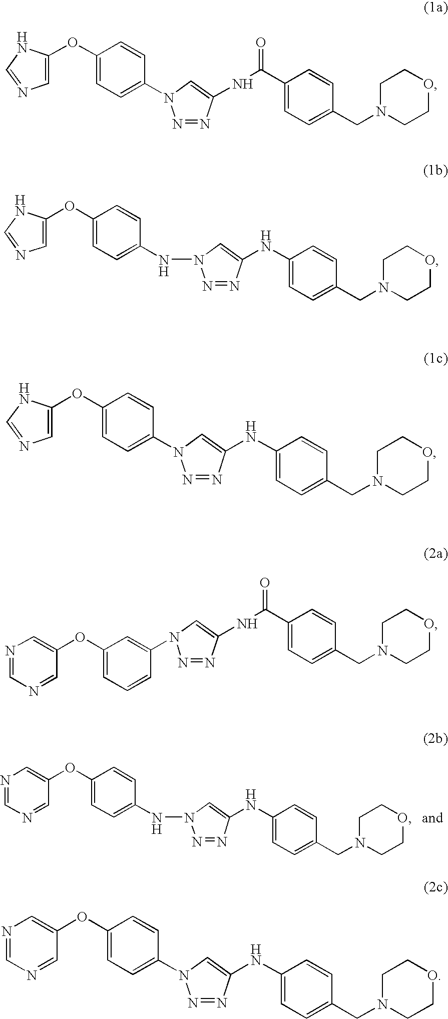

13. A pharmaceutical composition comprising
(a) the compound of claim 1;
(b) the pharmaceutical formulation of (a), comprising (formulated with) a cyclodextrin or a cycloamylose;
(c) the pharmaceutical formulation of (a) or (b), formulated as a sterile injectable aqueous solution, or an oleaginous suspension;
(d) the pharmaceutical formulation of any of (a) to (d), formulated with a (comprising a) diluent, an emulsifier, a preservative, a buffer, a pharmaceutically acceptable excipient, or a combination thereof; or
(e) the pharmaceutical formulation of any of (a) to (d), formulated as a liquid, an emulsion, a lyophilized powder, a spray, a cream, a lotion, a controlled release formulation, a tablet, a pill, a gel, a patch, in an implant or in a spray.

14. A pharmaceutical formulation comprising:
(a) the compound of claim 12;
(b) the pharmaceutical formulation of (a), comprising (formulated with) a cyclodextrin or a cycloamylose;
(c) the pharmaceutical formulation of (a) or (b), formulated as a sterile injectable aqueous solution, or an oleaginous suspension;
(d) the pharmaceutical formulation of any of (a) to (d), formulated with a (comprising a) diluent, an emulsifier, a preservative, a buffer, a pharmaceutically acceptable excipient, or a combination thereof; or
(e) the pharmaceutical formulation of any of (a) to (d), formulated as a liquid, an emulsion, a lyophilized powder, a spray, a cream, a lotion, a controlled release formulation, a tablet, a pill, a gel, a patch, in an implant or in a spray.

15. A liposome, a nanoparticle or a microsphere comprising the compound of claim 1.

16-19. (canceled)

20. A method for treating, preventing or ameliorating a disease or condition associated with dysfunctional cells, cancer stem cells or cancer cells activated by a DFG motif-comprising protein kinase, comprising
(1) (i) (a) providing the compound of claim 1; and,
(b) administering to an individual in need thereof an effective amount of the compound, pharmaceutical composition, pharmaceutical formulation or liposome, nanoparticle or microsphere;
(ii) the method of (i), wherein the individual is a human; or
(iii) the method of (i) or (ii), wherein the protein kinase is a B-raf, Lsk, c-Src, Fyn, Hck, Epha 3, Eprih A2, Ret, Fak, c-Met, ack1, c-kit, c-Fms, VEGF R2, FGFR1, IL1, EGFR2, Pak1, Ste20, TGFR-beta, c-abl or a Tak4 kinase; or
(2) the method of (1), wherein the cells, dysfunctional cells, cancer stem cells or cancer cells treated by the medicament are derived from or are cells or stem cells from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma or pituitary adenoma, and any combination thereof.

21. A method for treating, preventing or ameliorating a disease or condition associated with cells activated by a DFG motif-comprising protein kinase, comprising
(1) (i) (a) providing the compound of claim 1; and,
(b) administering to an individual in need thereof an effective amount of the compound, pharmaceutical composition, pharmaceutical formulation or liposome, nanoparticle or microsphere;
(ii) the method of (i), wherein the individual is a human; or
(iii) the method of (i) or (ii), wherein the protein kinase is a B-raf, Lsk, c-Src, Fyn, Hck, Epha 3, Eprih A2, Ret, Fak, c-Met, ack1, c-kit, c-Fms, VEGF R2, FGFR1, IL1, EGFR2, Pak1, Ste20, TGFR-beta, c-abl or a Tak4 kinase; or
(2) the method of (1), wherein the cells, dysfunctional cells, cancer stem cells or cancer cells treated by the medicament are derived from or are cells or stem cells from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma or pituitary adenoma, and any combination thereof.

22. (canceled)

23. A method for inhibiting a DGF motif-comprising protein kinase by disrupting or constraining a kinase-activating molecular rearrangement in the DGF motif, comprising
(1) (i) (a) contacting a DGF motif-comprising protein kinase with the compound of claim 1; or
(b) contacting a DGF motif-comprising protein kinase-comprising cell with the compound of claim 1;
(ii) the method of (i), wherein the contacting is in vitro, ex vivo, or in vivo; or
(iii) the method of (i) or (ii), wherein the protein kinase is a B-raf, Lsk, c-Src, Fyn, Hck, Epha 3, Eprih A2, Ret, Fak, c-Met, ack1, c-kit, c-Fms, VEGF R2, FGFR1, IL1, EGFR2, Pak1, Ste20, TGFR-beta, c-abl or a Tak4 kinase; or
(2) the method of (1), wherein the method ameliorates, treats or prevents a disease or condition comprising melanoma and colon cancer angiogenesis; hematopoeitic stem cell function; neurological disorders; cancer; angiogenesis; myeloproliferative disease; diabetes, hypertension, inflammation; Brutons immunodeficiency; neuro-regulator dysfunction, uremia induced hyperplasia, tumorigenesis, invasive metastasis, prostate cancer, breast cancer, colon cancer, pancreatic cancer, a leukemia; thrombosis; abnormal angiogenesis and/or a T cell related disease or condition; or
(3) wherein the kinase is selected from the group consisting of: B-raf, Lsk, c-Src, Fyn, Hck, Epha 3, Eprih A2, Ret, Fak, c-Met, ack1, c-kit, c-Fms, VEGF R2, FGFR1, IL1, EGFR2, Pak1, Ste20, TGFR-beta, c-abl and Tak4.

24. A method for inhibiting a kinase-activating molecular rearrangement in a DGF motif (interfering with the activation motion of a DFG switch), comprising:
(1) (a) contacting a DGF motif-comprising kinase with the compound of claim 1;
(b) the method of (a), wherein the contacting is in vitro, ex vivo or in vivo; or
(c) the method of (a) or (b), wherein the protein kinase is a B-raf, Lsk, c-Src, Fyn, Hck, Epha 3, Eprih A2, Ret, Fak, c-Met, ack1, c-kit, c-Fms, VEGF R2, FGFR1, IL1, EGFR2, Pak1, Ste20, TGFR-beta, c-abl or a Tak4 kinase; or
(2) the method of (1), wherein the method ameliorates, treats or prevents a disease or condition comprising melanoma and colon cancer angiogenesis; hematopoeitic stem cell function; neurological disorders; cancer; angiogenesis; myeloproliferative disease; diabetes, hypertension, inflammation; Brutons immunodeficiency; neuro-regulator dysfunction, uremia induced hyperplasia, tumorigenesis, invasive metastasis, prostate cancer, breast cancer, colon cancer, pancreatic cancer, a leukemia; thrombosis; abnormal angiogenesis and/or a T cell related disease or condition; or
(3) wherein the kinase is selected from the group consisting of: B-raf, Lsk, c-Src, Fyn, Hck, Epha 3, Eprih A2, Ret, Fak, c-Met, ack1, c-kit, c-Fms, VEGF R2, FGFR1, IL1, EGFR2, Pak1, Ste20, TGFR-beta, c-abl and Tak4.

25-26. (canceled)

27. A method for making the compound of claim 1 comprising the synthetic scheme of FIG. 6.

28. A method for making the compound of claim 12 comprising the synthetic scheme of FIG. 6.
